(19) United States
(12) Patent Application Publication
(10) Pub. No.: US 2026/0076578 A1
(43) Pub. Date: Mar. 19, 2026

(54) System and Method to Alter Heartbeats

(71) Applicant: Atlantis Limited Partnership, Marion, MA (US)

(72) Inventor: Edward BRAINARD, II, Marion, MA (US)

(21) Appl. No.: 19/327,905

(22) Filed: Sep. 12, 2025

Related U.S. Application Data
(60) Provisional application No. 63/695,520, filed on Sep. 17, 2024.

(51) Int. Cl. A61B 5/024; A61B 5/00; A61B 5/0205; A61B 5/0245; A61B 5/318

(52) U.S. Cl. CPC A61B 5/02405; A61B 5/0205; A61B 5/0245; A61B 5/318; A61B 5/486; A61B 5/7285; A61B 5/7405; A61B 5/742

(57) ABSTRACT

A system and method of training a subject to alter their heartbeat, including playing sounds with auditory prompts having a timing with a repeating pattern, and measuring pulses from the subject's heart to generate heart pulse measurements representing the subject's heartbeat. A visual representation of the heart pulse measurements is displayed, and the subject is guided to synchronize their heartbeat with the repeating pattern of the auditory prompts.

### CROSS-REFERENCE TO RELATED APPLICATION

This application claims priority to U.S. Provisional Application No. 63/695,520 filed on 17 Sep. 2024. The entire contents of the above-mentioned application are incorporated herein by reference as if set forth herein in entirety.

### FIELD OF THE INVENTION

This invention relates to enhancing synchronization of heartbeats with sounds utilizing a pulse sensor.

### BACKGROUND OF THE INVENTION

Heartbeats of a human heart exhibit a cardiac cycle from the beginning of one heartbeat to the beginning of the next heartbeat. The two ventricles of the heart generate the largest pressure pulse through the circulatory system, beginning with rapid contraction and pumping by the ventricles, referred to as systole. The heart muscle then relaxes and fills with blood, termed diastole. The two atria of the heart experience systole slightly before the ventricles contract, to assist filling of the ventricles prior to ventricular systole.

Blood surges in pulses through the circulatory system as pressure waves generated by systolic contractions. Each pulse can be felt directly by palpating arteries in certain regions of the body, or can be measured indirectly by observing the interaction of one or more wavelengths of light on or through the skin at those regions. The rate of pulses is close to the rate of heartbeats (heart rate) in healthy people, typically 60 to 100 beats per minute (BPM). The pulses can be observed in a photoplethysmogram (PPG) which is an optically obtained plethysmogram that detects blood volume changes in tissue due to cardiac pulses.

Devices such as pulse oximeters can be utilized to obtain PPGs. One type of pulse oximeter sends light from a LED (light emitting diode) right through a finger or through other parts of the subject's body, called a transmission or “transmissive” type. Another type reflects light off tissues of the body, such as off a finger, off the forehead or other anatomy, referred to as “reflectance”. Most pulse oximeters do not display a PPG, and instead calculate blood oxygenation and heart rate.

There are a number of irregular conditions of the heart, such as atrial fibrillation (“AFib”) or premature ventricular contractions (PVCs), that cause pulses to be irregular. These irregularities can be seen in a PPG. Treatment of heart irregularities typically involves administration of pharmaceuticals, surgical intervention, and/or implanting pacemakers. Such treatments can be expensive and invasive.

Different approaches that have been tried in the past include a method of presenting audible and visual cues to a human subject for the purpose of synchronizing breathing and heart rate variability as described in U.S. Pat. Nos. 7,255,672 and 7,497,821 by Elliott of Coherence LLC. Other approaches include an apparatus to measure a signal indicative of a user's biorhythmic activity and generate an output signal which directs the user to modify an action or physiological variable as disclosed by Gavish of Intercure Ltd. in U.S. Pat. No. 8,672,852. Methods purporting to control biometric parameters utilizing an audible tempo of music are discussed by LeBoeuf et al. in U.S. Pat. No. 11,058,304, for example.

It is therefore desirable to have alternative non-invasive techniques to improve heart function.

### SUMMARY OF THE INVENTION

An object of the present invention is to enable a user to alter their heartbeat by synchronizing their heartbeat with a repeating pattern of auditory prompts.

Another object of the present invention is to enable treatment of irregular heartbeats without resorting to pharmaceuticals or surgical interventions.

This invention features systems and methods of training a subject having a heart to alter their heartbeat, including playing sounds with auditory prompts having a timing with a repeating pattern, and measuring pulses from the subject's heart to generate heart pulse measurements representing the subject's heartbeat. A visual representation of the heart pulse measurements is displayed, and the subject is guided to synchronize their heartbeat with the repeating pattern of the auditory prompt.

In some embodiments, playing sounds includes playing music together with the auditory prompts. In a number of embodiments, guiding includes utilizing the heart pulse measurements to generate a heart pulse waveform including a selected portion of the waveform such as ventricular systole, also referred to herein as ventricular ejections, and generating and displaying an indication of synchronization between the selected portion of the waveform and the timing of the auditory prompts. In certain embodiments, the indication of synchronization includes determining pressure inflections such as minimums and/or maximums of the heart pulse waveform for a plurality of pulses to generate a pulse duration value “B” between a plurality of pressure inflections. A difference in timing between the auditory prompts and the pressure inflections is determined for each of a plurality of pressure inflections to generate a difference value “A”. The values of A and B are compared for each of the plurality of pulses, and a score based on the comparisons of A and B is displayed.

In a number of embodiments, measuring pulses includes observing the interaction of one or more wavelengths of light on or through a region of skin of the subject to detect blood volume changes in tissue underlying that region of skin. In some embodiments, measuring pulses includes measuring electrical impulses from the heart and generating an electrocardiogram. In certain embodiments, the method further includes encouraging the subject to alter a rate of breathing to assist synchronizing their heartbeat.

In some embodiments, at least one audio soundtrack has been recorded prior to playing sounds for the subject, with prompts added to at least one of the soundtracks. In certain embodiments, the prompts are based on tempo detected in music. A technician adds prompts to at least one soundtrack in some constructions and, in other constructions, prompts are automatically added to at least one track by auto-beat detection software.

### BRIEF DESCRIPTION OF THE DRAWINGS

To enable a better understanding of the present invention, and to show how the same may be carried into effect, certain embodiments of the invention are explained in more detail with reference to the drawings, by way of example only, in which:

FIG. 1 is a schematic block diagram of a system according to the present invention;

FIG. 2 is a schematic chart of a cardiac pressure pulse waveform plus prompts and analysis according to one embodiment of the present invention;

FIG. 3 is a graph showing heartbeats over time at various voltage readings from a sensor; and

FIG. 4 is a histogram generated for the heartbeats shown in FIG. 3.

### DETAILED DESCRIPTION OF THE PRESENTLY PREFERRED EMBODIMENTS

This invention may be accomplished by a system and method utilizing a photoplethysmogram (PPG) of a user (also referred-to herein as a “subject”) such as obtained from a pulse oximeter or other sensor that detects blood volume changes in tissue. Such sensors are referred to herein as “Pleth sensors”. Alternative constructions utilize electrical signals to generate an electrocardiogram (ECG) or utilize sound or ultrasound signals. Systems and methods according to the present invention utilize the pulse of a subject as measured in tissue of the subject, such as on the subject's finger, while the subject listens to sounds including auditory prompts. This technique is also referred to herein as Heart Timing Therapy or “HTT”. The subject views a representation of the pulse, and the method computes the magnitude of the pulse and applies one or more factors to it. In some constructions, the system generates a scoring number that goes from one to 2.00, so different people with different conditions can be tested and compared by relative scoring.

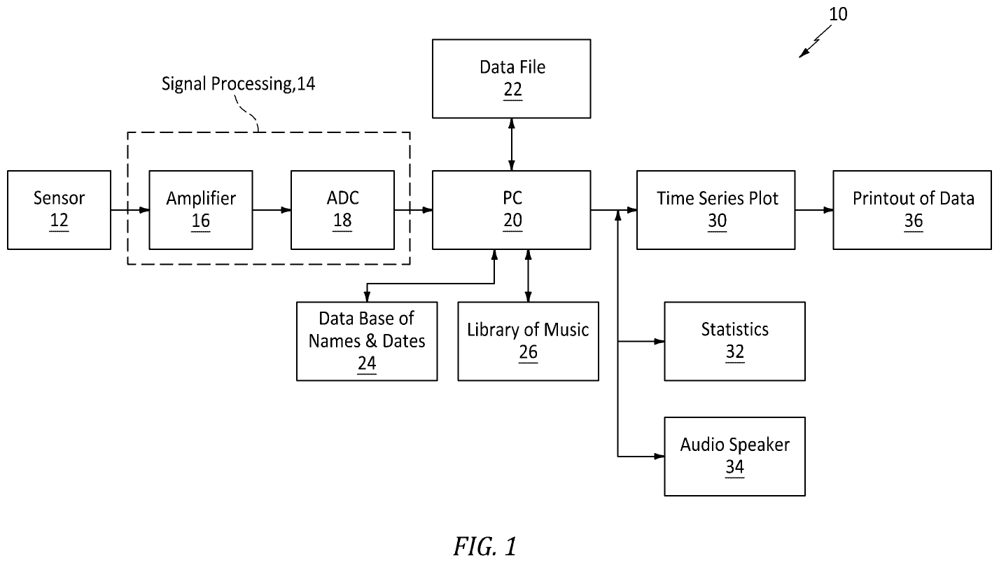

System 10 according to one construction of the present invention, FIG. 1, receives input from sensor 12 such as a Pleth sensor which generates analog pressure wave readings that are processed by signal processing 14 including an amplifier 16 and analog-to-digital converter ADC 18. Digitized pressure wave readings are provided to processor 20 such as a PC (Personal Computer) operating a program according to the present invention. As described in more detail below, processor PC 20 accesses a data file 22, a data base 24 of subject names and dates, and a library of music 26 which includes auditory prompts according to the present invention that are played on one or more audio speakers 34. Computations are made to generate a time series plot 30 viewable on a visual display, statistics 32, and a printout of data 36 or other recording of data generated by the present invention.

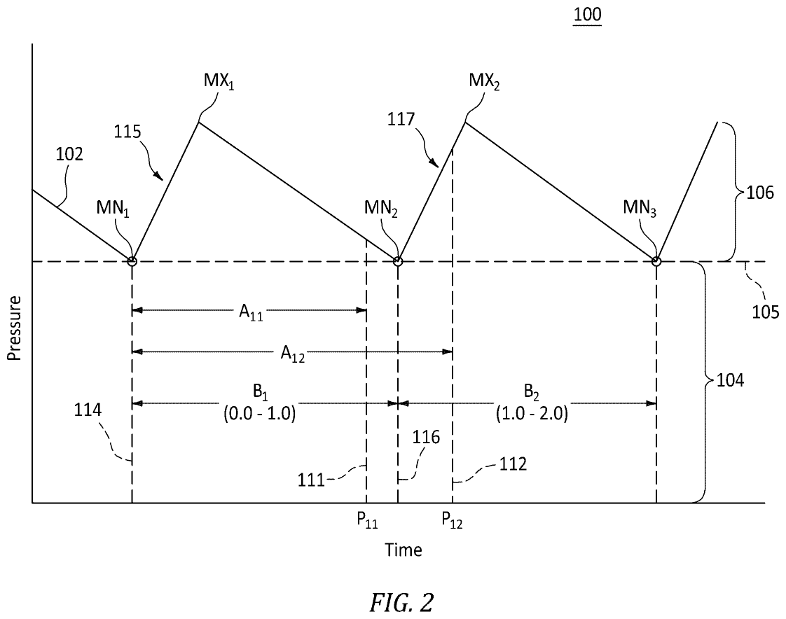

Chart 100, FIG. 2, depicts a cardiac pressure wave 102 having a DC steady pressure portion 104 and a variable AC pulsatile portion 106 that is utilized according to one construction of the present invention. AC portion 106 extends above horizontal dashed line 105 while DC portion 104 extends below dashed line 105. The y-axis of chart 100 relates to voltage representing blood pressure while time is indicated on the x-axis.

The term “portion” as utilized herein refers to a section or region of a component, without necessarily indicating any physical difference between two or more portions apart from location such as “upper portion” and “lower portion”. Qualifying terms such as “AC” and “DC” designate different characteristics for their respective portions 106 and 104 such as illustrated in FIG. 2.

For explanatory purposes, alternative single auditory prompts P₁₁ and P₁₂ according to the present invention are shown along the x-axis with dotted lines 111 and 112, respectively, in relation to pressure minimums MN₁ and MN₂ of pressure wave 102 in this construction. Analysis relative to pressure minimum MN₁ is described below. Pressure minimums MN₁ and MN₂ are one type of pressure inflection locatable on pressure wave curve 102; other pressure inflections utilized in other constructions include pressure maximums MX₁ and MX₂, for example, representing the end of a ventricular systolic phase and the beginning of a diastolic phase for respective heartbeats.

In this construction, the timing between MN₁ and MN₂ represents one cardiac cycle 115 of a subject and is designated to have a pulse duration value B₁. Dashed lines 114 and 116 represent the beginning of ventricular systolic phases, also referred to herein as ventricular ejections, for cardiac cycles 115 and 117 while MX₁ and MX₂ each represent the beginning of a ventricular diastolic phase. Similarly, pulse duration value B₂ begins at pressure minimum MN₂ of cardiac cycle 117 and continues until the next pressure minimum MN₃ is detected.

Also shown in FIG. 2 are a difference value A₁₁ extending between pressure minimum MN₁ and prompt P₁₁, and a difference value A₁₂ between pressure minimum MN₁ and prompt P₁₂ as explained in more detail below in relation to Equation 1. A pulse duration value B extends between two heartbeats so it's a time and, in one construction, the system assigns “1.0” for value B and then the distance from one pressure minimum to the prompt is A, so when A is divided by B a fraction is obtained. Alternative embodiments utilize percentages or other relationships. What this enables the system to do is rate how well a person is following the prompts and/or the music beat (tempo), and put a scoring number on it.

Returning to system 10, FIG. 1, operation in one construction proceeds. A pressure, sound, ultrasound or electrical sensor is placed on a subject. Voltage variations in the sensor caused by light changes from skin tissue variations caused by pulsate blood flow are passed to Signal Processing including AC signals amplified within Amplifier with 0.1-6 Hz. bandwidth passes signal to ADC (analog-to-digital converter). PC 20 requests Subject's name and guides subject to select a music file from Library 26 for which at least one audio soundtrack has been recorded prior to playing sounds for the subject, with prompts added to at least one of the soundtracks. In certain constructions, the prompts are based on tempo detected in music. A technician adds prompts in some constructions and, in other constructions, prompts are automatically added by “auto-beat detection” as described in more detail below. ADC 18 converts analog signal to digital data and feeds data to PC 20, which stores digitized heart pulse data to data file 22.

System starts upon initiation of an “HTT” program, and Sensor data is read at 500 Hz in one construction. Pre-recorded auditory prompts (Prompts) recorded in the music file are read and played with the selected music from audio file using the speaker. Prompts have a frequency within the detectable audio frequency range of humans, typically 20 Hz to 20,000 Hz (20 KHz).

In some constructions, the subject is instructed to follow music with their heartbeat using biofeedback with intentional or volitional breathing. The breathing cycle starts with inhaling generated by the muscle action within our chest cavity. Then, exhaling is driven by the contraction of our diaphragm, which drives the air out of our lungs.

Because the lungs make physical contact with the heart area, the cycling of the lung muscles physically modulates the heartbeat. Exhaling develops a thrust on the heart and provides extra pumping energy in our circulatory system during the diastolic phase of the heart pump cycle. This modulation can be felt in a relaxed state. Learning to control this thrust is the first step in learning biofeedback of the heart. By generating the thrust, you can control heart timing. When this practice is followed for long periods, it becomes more instinctive and happens almost automatically.

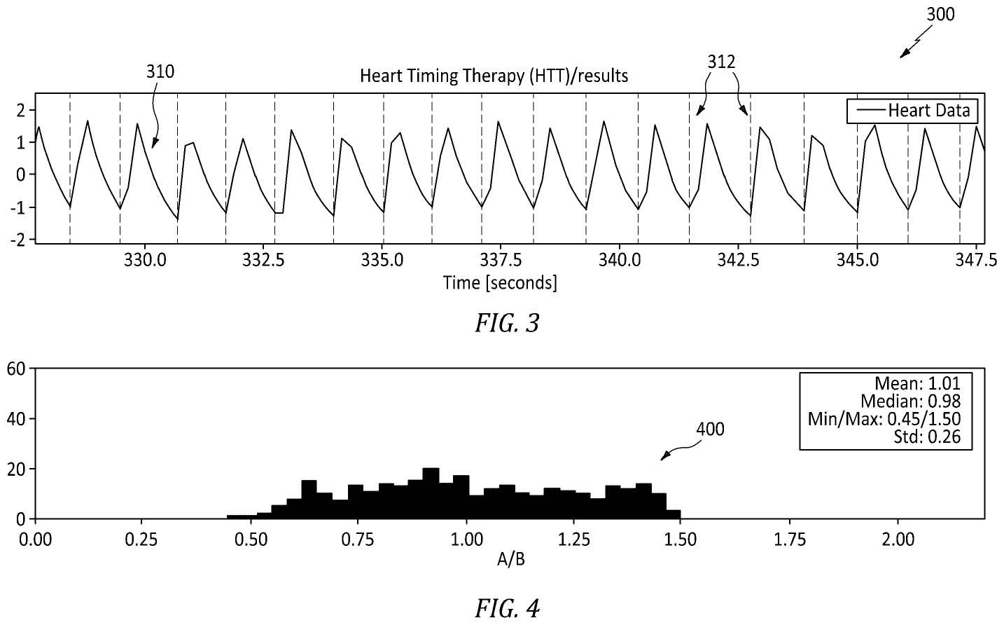

Histogram & Statistics will be displayed such as illustrated in FIGS. 3 and 4 during and/or at the end of the session. The data file will be automatically saved to a Documents folder in HTT Results. The document can be retrieved using the HTT Program “Open File” feature.

The subject or other authorized user then selects an HTT Results file in Desktop on PC 20, FIG. 1. A file is selected by the Subject's name or other identifying information, and the file is opened. The histogram and statistics will be presented, and can be saved as a plot file, or printed out to a printer.

The minimums of the heart pulse waveform are determined for a plurality of pulses to generate a pulse duration value “B” between a plurality of minimums. The difference in timing between the music Prompt and each heart minimum are determined for each of a plurality of minimums to generate a difference value “A”. In one construction the following pulse synchronous timing (PST) equation is solved:

$$PST = A / B \qquad Eq . 1$$

Utilizing the examples of difference value A₁₁ for prompt P₁₁ and value A₁₂ for prompt P₁₂ relative to pressure minimum MN₁ as illustrated in FIG. 2, prompt P₁₁ falls within pulse duration value B₁ and difference value A₁₁ is within the B₁ range of 0.0 to 1.0. By comparison, prompt P₁₂ occurs after MN₂ and lies within the span of B₂ and therefore has a value range of 1.0-2.0 relative to pressure minimum MN₁. Thus, Equation 1 can be utilized to generate a histogram such as histogram 400, FIG. 4, with values ranging between 0.00 and 2.00 as discussed in more detail below.

In one construction, data is plotted on a display screen such as shown for HTT Results 300, FIG. 3, with a Heart pulse wave form 310 and heart pulse minimum timing marked by the system with vertical dashed lines 312. Voltage readings from a sensor on the subject are indicated on the y-axis centered around zero. The subject listened to “Waltz of the Flowers” by Tchaikovsky at 65 BPM. A technician had previously added 1 KHz tonal prompts (3 cycles in duration) at 65 BPM. A histogram 400, FIG. 4, was developed pulse by pulse during the session and plotted on the display screen with Equation 1 results plotted along the x-axis. Statistics including PST from Equation 1 and beats per minute were computed on a running basis and plotted. At the end of the music file, program stops plotting and file of results is stored.

Scoring statistics calculated by the system utilizing Equation 1 above and displayed in FIG. 4 include a Mean of 1.01, a Median of 0.98, a Min/Max of 0.45-1.50 and a Standard Deviation of 0.26. In this construction, scoring is based on heartbeat timing relative to prompts, rather than signal amplitude.

Prompts according to the present invention typically range between 60 BPM-120 BPM, more preferably 65 BPM-100 BPM. These prompts were added to dance music including waltzes, tangos, foxtrot and samba. Other types of music having tempos within 60 BPM-120 BPM can be utilized according to the present invention. In other constructions, a metronome beat is added as the prompts according to the present invention.

Detecting the beat in music in real-time to have the music beat serve as prompts according to the present invention involves analyzing the audio signal to identify periodic patterns or rhythmic elements that correspond to beats. Existing methods and techniques which can be utilized for “auto-beat detection” include (1) Onset Detection, (2) Tempo Estimation and Beat Tracking, (3) Machine Learning-based Beat Detection, (4) Phase Locking and Synchronization, and (5) Signal Filtering and Smoothing. Onset Detection focuses on identifying the onset of musical notes or events, which often coincide with the beats. Onset detection algorithms analyze the audio signal for sudden changes in amplitude or frequency content. Techniques include energy-based onset detection and spectral-based onset detection. Energy-based approaches monitor the energy of the signal over time and detects peaks where there is a sudden increase in energy, indicating the start of a beat. Spectral-based approaches track changes in the spectral content of the audio signal. Sudden shifts in the frequency domain often indicate the presence of a new musical event, such as a beat.

Once the onset times are detected, tempo estimation methods analyze the periodicity of these events to determine the overall tempo in beats per minute (BPM) of the music. Beat tracking then aligns beats to the estimated tempo. Techniques include autocorrelation methods that calculate the similarity of a signal to a time-shifted version of itself. Peaks in the autocorrelation function indicate periodic patterns that correspond to the beat interval. Using Fourier transform, the signal is converted into the frequency domain, where periodic components that correspond to the tempo can be identified. Alternatively, dynamic programming optimizes the alignment of detected onsets to the estimated beat grid by minimizing errors in beat placement over time.

Machine learning models, especially deep learning techniques, can be trained to detect beats in real-time by learning patterns from labelled datasets. These models can capture more complex relationships in the music. Techniques include Convolutional Neural Networks (CNNs) that can process spectrograms of audio signals to detect beats based on learned features. Alternatively, Recurrent Neural Networks (RNNs) can handle sequential data and can be used to track beats by learning temporal dependencies in music. Some models combine onset detection with machine learning to improve accuracy, especially in complex or polyphonic music.

Phase Locking and Synchronization involves aligning the detected beats to a regular grid (e.g., a metronome). This method is useful for synchronizing music playback or other time-dependent applications. A Phase-locked Loop (PLL) control system adjusts the detected beat phase to align with a reference clock or grid, ensuring consistent synchronization. Alternatively, Kalman Filters can be utilized to track the beat phase and tempo in noisy or unpredictable environments, updating predictions as new audio data comes in.

Finally, Signal Filtering and Smoothing utilizes various filtering techniques to smooth out the detected beats and reduce noise to enhance stability and accuracy of beat detection. Techniques include Low-pass Filtering to remove high-frequency noise that could cause false positives in beat detection. A Moving Average Filter smooths the detected beat times by averaging over a short window, reducing jitter in the beat positions.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is to be understood that the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions. Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on, or executable by, a programmable computer including any combination of any number of the following: a processor, a storage medium readable and/or writable by the processor (including, for example, volatile and non-volatile memory and/or storage elements), an input device, and an output device. The input device and/or the output device form a user interface in some embodiments. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention automatically compares the timing of each heartbeat with a prompt to generate performance scoring, automatically updates data in an electronic memory representing such scoring, and can display and/or wirelessly transmit such data to a server over a digital electronic network for storage and processing. Such features can only be performed by computers and other machines and cannot be performed manually or mentally by humans.

Any claims herein which affirmatively require a computer, a processor, a controller, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a controller, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays).

A computer can generally also receive (read) programs and data from, and write (store) programs and data to, a non-transitory computer-readable storage medium such as an internal disk (not shown) or a removable disk or flash memory. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium or other type of user interface. Any data disclosed herein may be implemented, for example, in one or more data structures tangibly stored on a non-transitory computer-readable medium. Embodiments of the invention may store such data in such data structure(s) and read such data from such data structure(s).

It is the intention, therefore, to be limited only as indicated by the scope of the claims appended hereto. Other embodiments will occur to those skilled in the art after reviewing the present disclosure and are within the following claims.

What is claimed is:

1. A method of training a subject having a heart to alter their heartbeat, comprising:
playing sounds including auditory prompts having a timing with a repeating pattern;
measuring pulses from the subject's heart to generate heart pulse measurements representing the subject's heartbeat;
displaying a visual representation of the heart pulse measurements; and
guiding the subject to synchronize their heartbeat with the repeating pattern of the auditory prompts.

2. The method of claim 1 wherein playing sounds includes playing music together with the auditory prompts.

3. The method of claim 1 wherein guiding includes utilizing the heart pulse measurements to generate a heart pulse waveform including a selected portion of the waveform, and generating and displaying an indication of synchronization between the selected portion of the waveform and the timing of the auditory prompts.

4. The method of claim 3 wherein the indication of synchronization includes determining pressure inflections of the heart pulse waveform for a plurality of pulses to generate a pulse duration value “B” between a plurality of pressure inflections.

5. The method of claim 4 further including determining a difference in timing between the auditory prompts and the pressure inflections for each of a plurality of pressure inflections to generate a difference value “A”.

6. The method of claim 5 further including comparing the values of A and B for each of the plurality of pulses, and displaying a score based on the comparisons of A and B.

7. The method of claim 1 wherein measuring pulses includes observing the interaction of one or more wavelengths of light on or through a region of skin of the subject to detect blood volume changes in tissue underlying that region of skin.

8. The method of claim 1 wherein measuring pulses includes measuring electrical impulses from the heart and generating an electrocardiogram.

9. The method of claim 1 further including encouraging the subject to alter a rate of breathing to assist synchronizing their heartbeat.

10. The method of claim 1 further including recording at least one audio soundtrack prior to playing sounds for the subject, and adding prompts to the soundtrack.

11. The method of claim 10 wherein the prompts are based on tempo detected in music.

12. The method of claim 6 wherein at least one histogram is displayed based on the comparison of A and B for each of a plurality of heartbeats of the subject.

13. A method of training a subject having a heart to alter their heartbeat, comprising:
playing pre-recorded sounds including music with auditory prompts having a timing with a repeating pattern;
measuring pressure pulses from the subject's heart to generate heart pulse measurements representing the subject's heartbeat;
displaying a visual representation of the heart pulse measurements and the auditory prompts; and
guiding the subject to synchronize their heartbeat with the repeating pattern of the auditory prompts, wherein guiding includes utilizing the heart pulse measurements to generate a heart pulse pressure waveform including ventricular systole, and generating and displaying an indication of synchronization between the beginning of ventricular systole and the timing of the auditory prompts.

14. The method of claim 13 wherein the indication of synchronization includes determining pressure minimums of the heart pulse waveform for a plurality of pulses to generate a pulse duration value “B” between a plurality of pressure minimums, and further including determining a difference in timing between the auditory prompts and the pressure minimum for each of a plurality of minimums to generate a difference value “A”.

15. The method of claim 14 further including comparing the values of A and B for each of the plurality of pulses, and displaying a score based on the comparisons of A and B.

16. The method of claim 15 further including recording at least one audio soundtrack prior to playing sounds for the subject, and adding prompts to the soundtrack.

17. The method of claim 16 wherein the prompts are based on tempo detected in music.

18. A method of treating a subject with a condition associated with irregular heartbeat, comprising:
playing sounds including the auditory prompts having a timing with a repeating pattern;
measuring a pulse of the subject;
displaying a visual representation of the pulse measurement and the auditory prompts; and
training the subject to synchronize their pulse with the auditory prompts using the visual representation, whereby the subject is trained to self-control their pulse when the condition associated with the irregular heartbeat arises.

19. The method according to claim 18, wherein training the subject includes providing an indication of synchronization between the pulse of the subject with the auditory prompts.

20. The method according to claim 19, wherein training the subject includes providing an indication that synchronization between a rate of breathing of the subject and the pulse of the subject with auditory prompts is within a predetermined threshold indicating success.

21. A system for treating a subject with a condition associated with an irregular heartbeat, comprising:
a sensor for measuring a pulse of the subject;
a speaker for receiving an audio signal and outputting an audible sound;
a display; and
a processor configured to:
provide the audio signal to the speaker, the audio signal including auditory prompts having a timing with a repeating pattern;
receive the pulse measurement from the sensor;
display a visual representation of the pulse measurement and the auditory prompts; and
train the subject to synchronize their pulse with the auditory prompts using the visual representation, whereby that the subject is trained to self-control their pulse when the condition with the irregular heartbeat arises.

22. The system according to claim 21, wherein the processor is configured to provide an indication of synchronization between the pulse of the subject with the auditory prompts.

23. The system according to claim 21, wherein the processor is configured to provide an indication that synchronization between a rate of breathing of the subject and pulse of the subject with auditory prompts is within a predetermined threshold indicating success.
